(19) United States
(12) Patent Application Publication
(10) Pub. No.: US 2004/0097923 A1
(43) Pub. Date: May 20, 2004

(54) Fluidic compression device adapted to accommodate an external fixation device

(75) Inventor: Daniel P. Shevlin, McKinney, TX (US)

Correspondence Address: BAKER BOTTS L.L.P., 2001 ROSS AVENUE, SUITE 600, DALLAS, TX, 75201-2980, US

(73) Assignee: EEMSO, Inc.

(21) Appl. No.: 10/619,728

(22) Filed: Jul. 15, 2003

Related U.S. Application Data
(60) Provisional application No. 60/396,523, filed on Jul. 17, 2002.

(51) Int. Cl. A61B 17/58

(52) U.S. Cl. 606/54

(57) ABSTRACT

In certain embodiments, a fluidic compression device adapted to accommodate an external fixation device includes at least one fluid-impermeable bladder adapted to contain a fluid and at least one region adapted to receive and accommodate one or more external portions of an external fixation device. The fluidic compression device is adapted to be positioned about a portion of a user's body such that an opening formed within the region receives and accommodates one or more external portions of the external fixation device. The fluidic compression device is further adapted to apply intermittent compression to the portion of the user's body according to intermittent increases and decreases in fluid pressure in the bladder while the device is positioned about the portion of the user's body and is accommodating the external fixation device.

### RELATED APPLICATIONS

This application claims the benefit under 35 U.S.C. §119(e) of U.S. Provisional Patent Application No. 60/396,523 entitled “Fluodic Compression Apparatus Accommodating An External Fixation Device” filed Jul. 17, 2002.

### TECHNICAL FIELD OF THE INVENTION

This invention relates generally to medical devices and, more particularly, to a fluidic compression device adapted to accommodate an external fixation device.

### BACKGROUND

Fluidic compression devices may be used to prevent or treat deep venous thrombosis (DVT) (i.e. blood clots in the legs), pulmonary embolism (PE) (i.e. blood clots in the lungs), or other circulation problems. For example, such problems may arise following surgery or other trauma to a limb. If such problems are not prevented or are left untreated the consequences are potentially fatal. Using a fluidic compression device that has been filled with air, water, or another suitable fluid, pressure may be periodically, cyclically, or otherwise intermittently applied to a portion of a user's body, such as the ankle or another portion of the leg or other limb, to increase venous blood flow and thereby prevent or treat DVT, PE, or other circulation problems.

### SUMMARY OF THE INVENTION

According to the present invention, certain disadvantages and problems associated with previous fluidic compression devices may be reduced or eliminated.

In certain embodiments, a fluidic compression device adapted to accommodate an external fixation device includes at least one fluid-impermeable bladder adapted to contain a fluid and at least one region adapted to receive and accommodate one or more external portions of an external fixation device. The fluidic compression device is adapted to be positioned about a portion of a user's body such that an opening formed within the region receives and accommodates one or more external portions of the external fixation device. The fluidic compression device is further adapted to apply intermittent compression to the portion of the user's body according to intermittent increases and decreases in fluid pressure in the bladder while the device is positioned about the portion of the user's body and is accommodating the external fixation device.

In other embodiments, a method for manufacturing a fluidic compression device adapted to accommodate an external fixation device includes providing at least one fluid-impermeable bladder adapted to contain a fluid and providing at least one region adapted to receive and accommodate one or more external portions of an external fixation device. The fluidic compression device is adapted to be positioned about a portion of a user's body such that an opening formed within the region receives and accommodates the one or more external portions of the external fixation device. The fluidic compression device is further adapted to apply intermittent compression to the portion of the user's body according to intermittent increases and decreases in fluid pressure in the bladder while the device is positioned about the portion of the user's body and is accommodating the external fixation device.

In other embodiments, a method for preventing or treating circulation problems includes positioning a fluidic compression device about a portion of a user's body such that at least one opening formed in a region of the device receives and accommodates one or more external portions of an external fixation device. The method further includes at least partially filling at least one fluid-impermeable bladder of the fluidic compression device with fluid. The method further includes intermittently increasing and decreasing fluid pressure within the bladder to apply intermittent compression to the portion of the user's body while the fluidic compression device is positioned about the portion of the user's body and is accommodating the external fixation device, the intermittent compression facilitating prevention or treatment of circulation problems associated with the portion of the user's body.

Particular embodiments of the present invention may provide one or more technical advantages. Certain embodiments provide a fluidic compression device that accommodates an external fixation device that is already in place with respect to a portion of a user's body, such as the user's ankle or another portion of the user's leg or other limb. Certain embodiments allow suitable pressure to be periodically, cyclically, or otherwise intermittently applied to the portion of the user's body to prevent or treat DVT, PE, or other circulation problems. For example, such problems often arise following surgery or other significant trauma to the portion of the user's body due to post-operative immobilization, including external fixation, and concomitant loss of venous pump function, which may allow blood clots to form. It has been approximated that forty percent of users receive external fixation devices following limb surgery. However, despite the risks of DVT, PE, or other circulation problems and the potentially fatal consequences of such problems, previous fluidic compression devices have not accommodated external fixation devices, leaving a significant portion of the at risk population unserved. Certain embodiments may be readily customized to accommodate a particular external fixation device. Certain embodiments allow customization to be performed without jeopardizing the integrity of the bladder within the device containing the fluid. Certain embodiments accommodate a variety of external fixation devices, and preferably any suitable external fixation device, with or without customization.

Certain embodiments may provide all, some, or none of these technical advantages. Certain embodiments may provide one or more other technical advantages, one or more of which may be readily apparent to those skilled in the art from the figures, description, and claims included herein.

### BRIEF DESCRIPTION OF THE DRAWINGS

To provide a more complete understanding of the present invention and the features and advantages thereof, reference is made to the following description taken in conjunction with the accompanying drawings, in which:

FIG. 1 illustrates an example fluidic compression device in use in combination with an external fixation device;

FIG. 2 illustrates a side view of an example fluidic compression device;

FIG. 3 illustrates a side view of an example fluidic compression device including at least one fluid-impermeable bladder;

FIG. 4 illustrates a side view of a portion of an example fluidic compression device including a sealed cut line;

FIG. 5 illustrates an example connector;

FIG. 6 illustrates a cross-sectional view of an example fluidic compression device;

FIGS. 7A and 7B illustrate side views of alternative embodiments of a strap of an example fluidic compression device; and

FIG. 8 illustrates an example method for preventing or treating circulation problems using an example fluidic compression device.

### DESCRIPTION OF EXAMPLE EMBODIMENTS

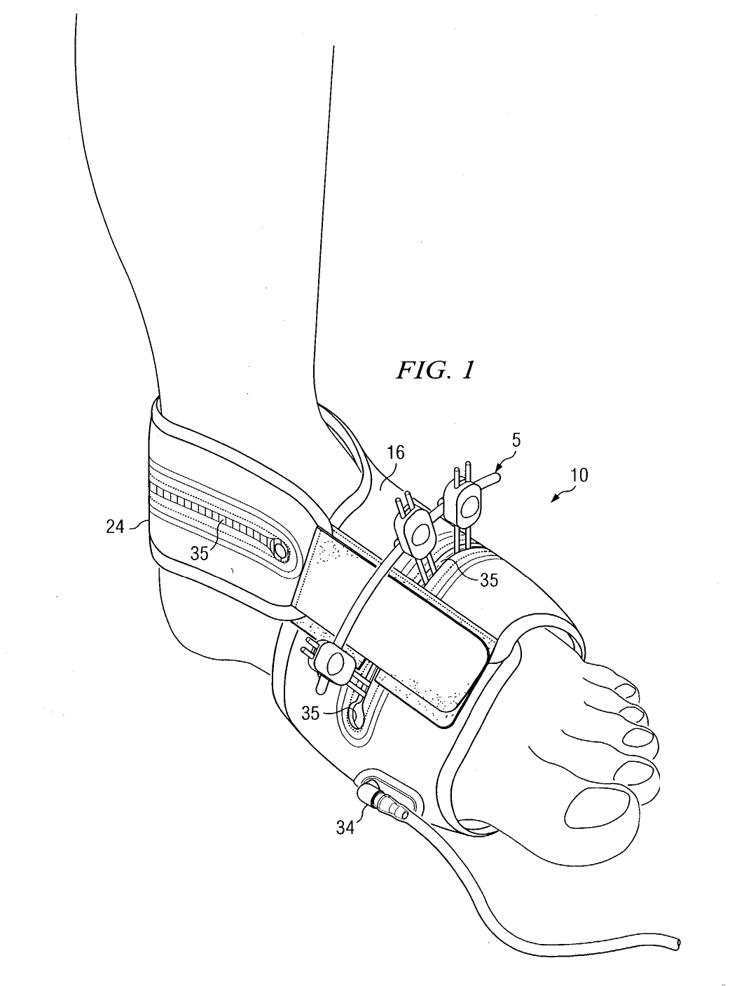

FIG. 1 illustrates an example fluidic compression device 10 in use in combination with an external fixation device 5 on a user's foot and ankle. In general, device 10 may be used to periodically, cyclically, or otherwise intermittently apply pressure to a portion of a user's body while accommodating an external fixation device 5 already in place on the user's body to prevent or treat DVT, PE, or other circulation problems. Although device 10 for fluidic compression of portions of a user's foot and ankle is illustrated by way of example, the present invention contemplates device 10 for fluidic compression of any suitable portion of the user's body while accommodating an external fixation device 5. Furthermore, although the use of device 10 in association with an external fixation device 5 is illustrated, the present invention contemplates device 10 being used to benefit the user whether or not an external fixation device 5 is also being used. The ability to accommodate an external fixation device 5 may provide an important technical advantage. In certain embodiments, device 10 may be readily customized, in the manner described below for example, to accommodate a particular external fixation device 5. In certain embodiments, device 10 may accommodate a variety of external fixation devices 5, and preferably any suitable external fixation device 5, with or without customization.

Device 10 may be constructed using a soft, foldable, and flexible external woven material that at least substantially covers a soft, foldable, and flexible internal foam material. These materials may encompass one or more silastic or other fluid-impermeable bladders that may be at least partially filled with fluid during use. As used in this document, the term “fluidic” is meant to encompass use of any suitable fluid within device 10, such as air, water, or any other fluid. The pressure of the fluid contained in the bladder may be cycled to apply compression to a portion of the user's body, such as an ankle or another portion of the leg or other limb, to increase venous blood flow and thereby prevent or treat DVT, PE, or other circulation problems. In a particular embodiment, when the bladder is at least partially filled with fluid, device 10 may apply approximately one hundred thirty millimeters of mercury (130 mm Hg) pressure to approximately one hundred eighty millimeters of mercury (180 mm Hg) pressure to the portion of the user's body being compressed. Although an example fluid pressure range is provided, the present invention contemplates any appropriate pressure and pressure range according to particular needs and circumstances. For example, the fluid pressure and fluid pressure range for treatment of a foot and ankle may be different than the fluid pressure and fluid pressure range for treatment of a leg, arm, or other limb.

In certain embodiments, fluidic compression device 10 includes a circumferential portion 16 that wraps around portions of the user's foot and ankle. Device 10 may also include a heel strap portion 24 that wraps around portions of the user's heel. Device 10 may include one or more openings 35 each adapted to receive and accommodate one or more corresponding external portions of external fixation device 5. As described more fully below, in certain embodiments each such opening 35 may be cut within a sealed region to customize device 10 for a particular external fixation device 5. In certain embodiments, openings 35 accommodate a variety of external fixation devices 5, and preferably any suitable external fixation device 5, with or without customization.

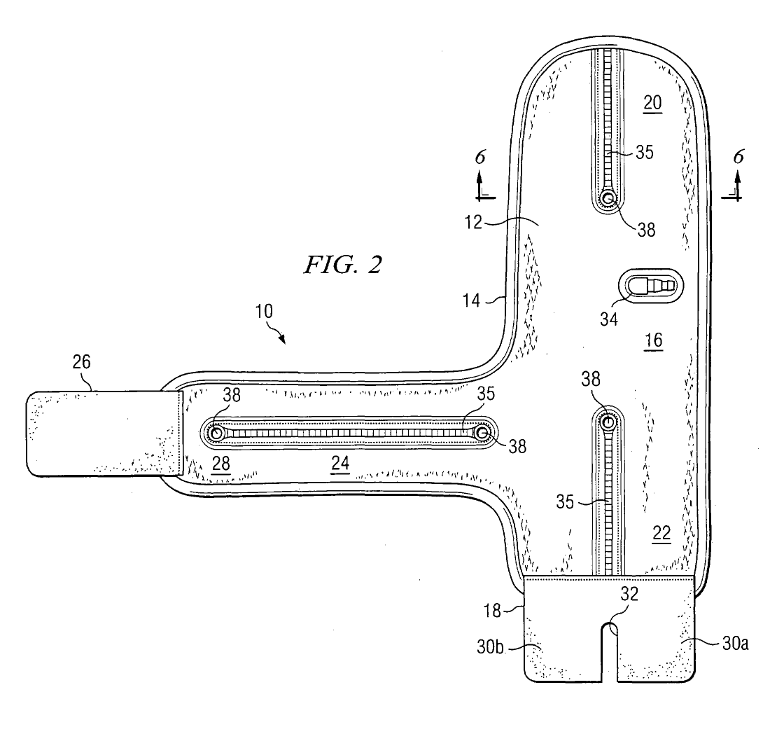

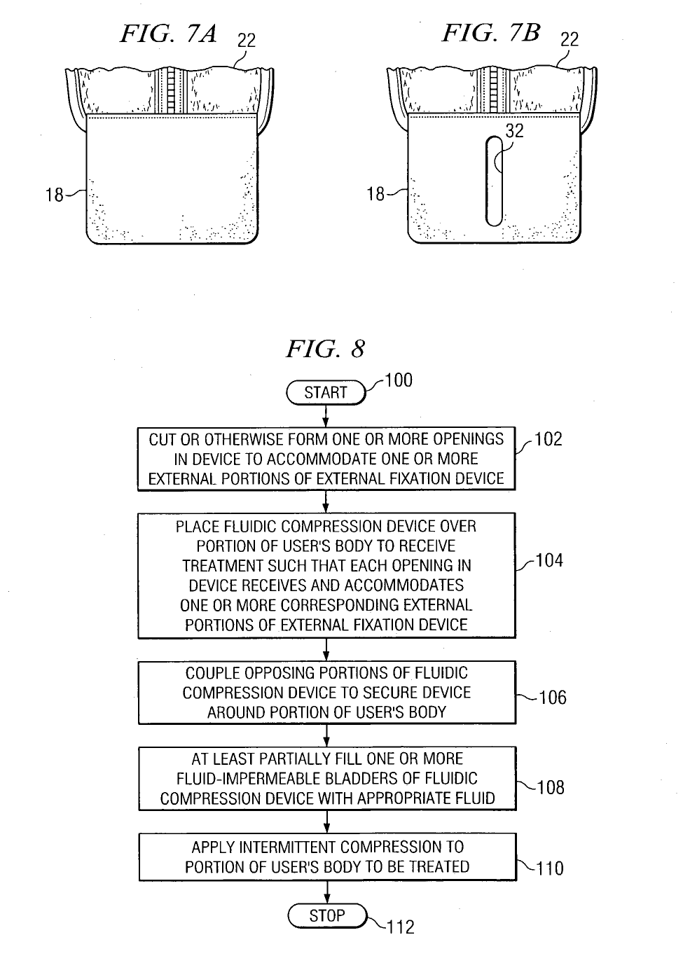

FIG. 2 illustrates a side view of an example fluidic compression device 10. In certain embodiments, fluidic compression device 10 includes a main body 12 and an edge 14 that substantially surrounds main body 12. A cross-sectional view of edge 14 and main body 12 is discussed below with reference to FIG. 6. Circumferential portion 16 may include a strap 18 used to couple an elongated lateral portion 20 of circumferential portion 16 to a medial portion 22 of circumferential portion 16 to secure device 10 around portions of the user's foot and ankle. In certain embodiments, strap 18 may include two separate portions 30a and 30b allowing strap 18 to be fitted around a variety of external fixation devices 5, and preferably any suitable external fixation device 5, which may be passed through strap 18 within an opening 32. Alternative embodiments of strap 18 are discussed below with reference to FIGS. 7A and 7B. Heel strap portion 24 may include a strap 26 used to couple an elongated end portion 28 of heel strap portion 24 to circumferential portion 16 to secure device 10 around portions of the user's heel and foot.

Straps 18 and 26 are preferably, but need not be, substantially inelastic so as to provide limited lengthening or widening under stress. Straps 18 and 26 may, with corresponding locations of elongated lateral portion 20 of circumferential portion 16 and elongated end portion 28 of heel strap portion 24, respectively, support any appropriate fasteners. For example, in certain embodiments, straps 18 and 26 support VELCRO or another suitable hook and loop fastener to secure straps 18 and 26 to corresponding locations on elongated lateral portion 20 of circumferential portion 16 and elongated end portion 28 of heel strap portion 24, respectively. In a particular embodiment, VELCRO 3600 or VELCRO 3800 may be used. However, any suitable fasteners, such as snaps, buttons, or other fasteners, may be used according to particular needs.

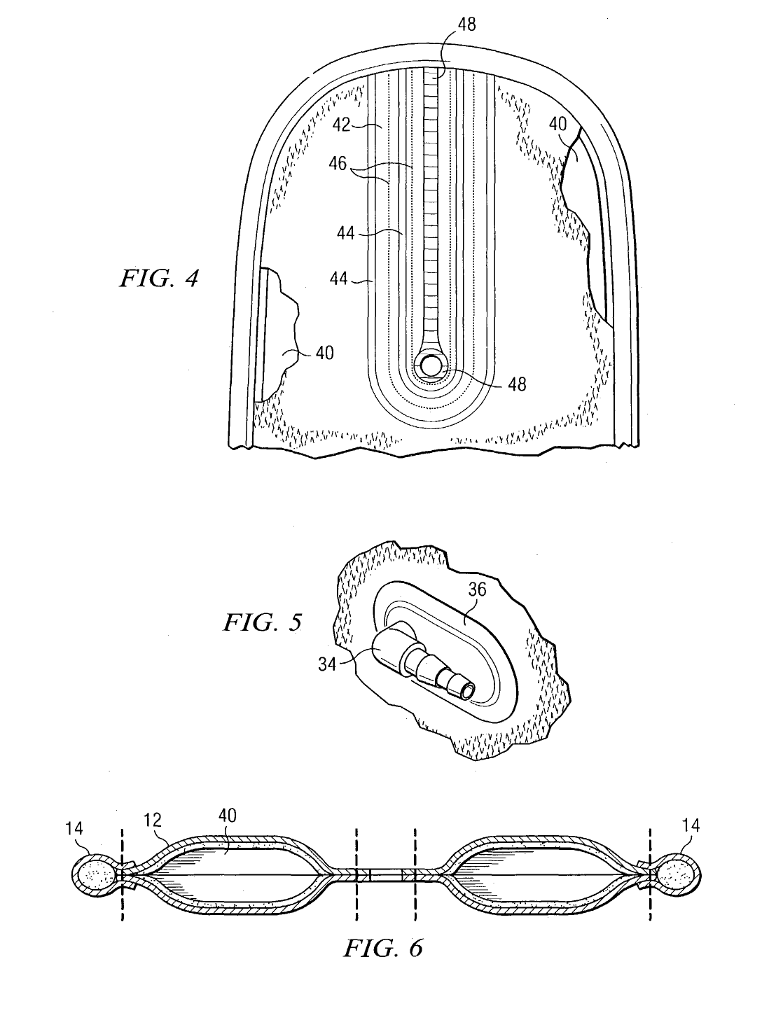

Device 10 may include a connector 34 for coupling device 10 to a fluid source and for receiving air, water, or another suitable fluid from the fluid source to at least partially fill the bladder of device 10 and increase the fluid pressure within the bladder as desired. Connector 34 may also be used to release fluid from the bladder to decrease the fluid pressure within the bladder as desired. Through intermittent increases and decreases in the fluid pressure within the bladder, intermittent compression of the targeted portion of the user's body is achieved. An example connector 34 is described in more detail below with reference to FIG. 5.

In certain embodiments, as described above, device 10 is customizable to accommodate a particular external fixation device 5 using one or more openings 35. Device 10 may include pre-formed holes 38 that are preferably sized to accommodate a cutting device. The cutting device may be inserted in a hole 38 and then used to cut through device 10 to form an opening 35 sufficient to receive and accommodate one or more corresponding external portions of the particular external fixation device 5. As discussed below with reference to FIG. 4, in certain embodiments, a visible cut line may be provided on device 10 to guide such cutting. Although four preformed holes 38 and associated openings 35 in particular locations are illustrated by way of example, the present invention contemplates any suitable number of holes 38 and associated openings 35 in any suitable locations.

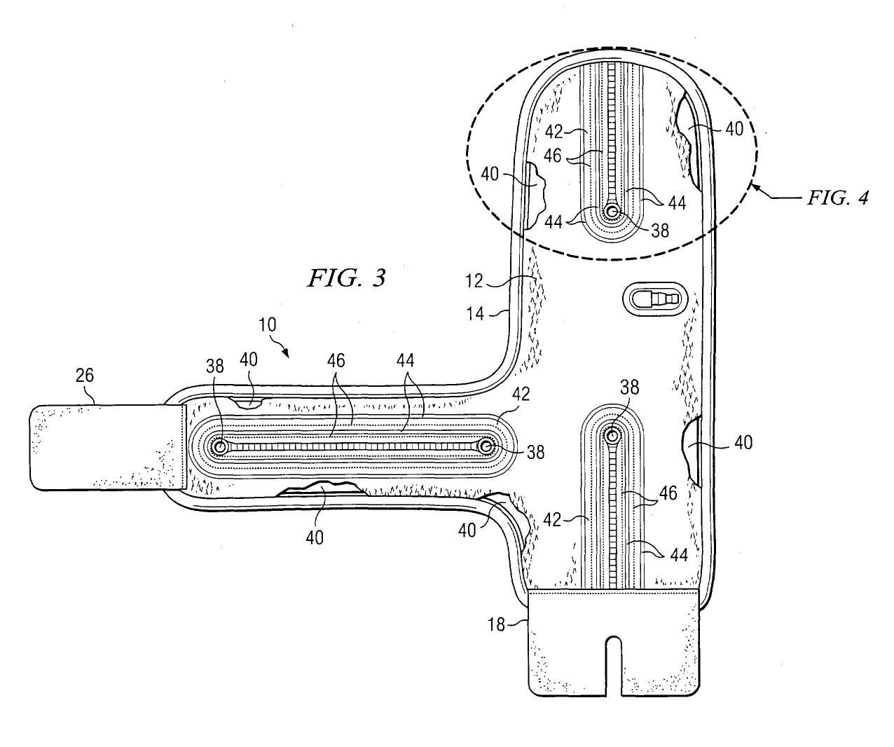

FIG. 3 illustrates a side view of an example fluidic compression device 10 including a fluid-impermeable bladder 40. One or more such bladders 40 may be provided within device 10. In certain embodiments, fluidic compression device 10 may include one or more seals 44, which define one or more sealed regions 42. Seals 44 may comprise hermetic seals, radio frequency (RF) seals, or any other appropriate seal. Double seals 44 may be provided for increased durability and reliability. Device 10 may also include stitching 46 near each seal 44 within the corresponding sealed region 42 to provide additional durability and reliability. Stitching 46 may also provide a safety margin during cutting of an opening 35. Stitching 46, in combination with seals 44, may help to ensure that device 10 can be cut as necessary to accommodate any suitable external fixation device 5 without comprising the integrity of bladder 40. Seals 44 and stitching 46 may therefore serve as an “integrity buffer” for bladder 40. In certain embodiments, bladder 40 provides a single fluid-impermeable chamber, shaped according to the configuration of sealed regions 42, which substantially fills the surrounding regions of device 10. However, as described above, device 10 may include one or more bladders 40 providing one or more such chambers.

FIG. 4 illustrates a side view of a portion of an example fluidic compression device 10 including a sealed cut line 48. As discussed above, a user may cut through device 10 within a sealed region 42 to form an opening 35 in device 10 sufficient to receive one or more external portions of external fixation device 5. In certain embodiments, cut line 48 may be indicated visibly on device 10, such as by a solid or dotted line printed on device 10, and may also be perforated to allow cutting to be performed more easily. Cut line 48 may be used to guide a user in cutting within sealed region 42 such that bladder 40 is not perforated during cutting.

FIG. 5 illustrates an example connector 34. In certain embodiments, connector 34 may be a graduated, multi-circumference connector for coupling to an appropriate fluid source and for receiving air, water, or another suitable fluid from the fluid source to at least partially fill bladder 40. For example, connector 34 may couple to a hose to receive air from a pneumatic compression pump. Connector 34 may also be operable to release fluid from bladder 40. In a particular embodiment, connector 34 may include a valve to prevent fluid pressure from unintentionally escaping from bladder 40 after bladder 40 is filled with an appropriate fluid. Through intermittent increases and decreases in the fluid pressure within bladder 40, intermittent compression of the targeted portion of the user's body is achieved. A flange 36 may be used to support connector 34 and prevent leakage of fluid from bladder 40. In certain embodiments, flange 36 may be formed from a hardened silastic, plastic, or other suitable material and may be formed in a “stair-stepped” configuration. Flange 36 may be sealed in two locations for improved durability and stronger adherence to main body portion 12 during installation and use.

FIG. 6 illustrates a cross-sectional view of an example fluidic compression device 10. As described above, device 10, including main body 12 and edge 14, may be constructed using a soft, foldable, and flexible external woven material that at least substantially covers a soft, foldable, and flexible internal foam material. In main body 12, these materials encompass one or more silastic or other fluid-impermeable bladders 40 that may be at least partially filled with fluid during use. FIG. 6 shows bladders 40 in an inflated state. In a particular embodiment, the external woven material of main body 12 may be sealed, bonded, stitched, or otherwise coupled to the internal foam and the internal foam may be sealed, bonded, stitched, or otherwise coupled to the one or more bladders 40.

A combination of the same external woven material and internal foam material used to form main body 12 may also be configured to form a “rolled” or otherwise rounded edge 14 to improve comfort and to prevent or reduce problems such as dermal ulcerations. For example, the external woven material may be rolled around the internal foam material and sealed, bonded, stitched, or otherwise coupled to main body portion 12. In a particular embodiment, the external woven material of edge 14 may be sealed, bonded, stitched, or otherwise coupled to the internal foam material. In FIG. 6, the external woven material of main body 12 and rounded edge 14 is indicated using diagonal stripes and the internal foam material is indicated using stippling.

FIGS. 7A and 7B illustrate alternative example straps 18. In certain embodiments, as shown in FIG. 7A, strap 18 may be provided without any cutouts or openings 32. In another embodiment, as shown in FIG. 7B, strap 18 may be provided with an enclosed opening 32 formed in strap 18, allowing strap 18 to be fitted around a variety of external fixation devices 5, and preferably any suitable external fixation device 5, which may pass through strap 18 within opening 32.

FIG. 8 illustrates an example method for using device 10 to prevent or treat DVT, PE, or other circulation problems. The example method begins at step 102, when one or more openings 35 are cut or otherwise formed in device 10 to accommodate one or more external portions of an external fixation device 5. As described above, in certain embodiments, device 10 may be customized in this manner to accommodate any suitable external fixation device 5. At step 104, device 10 is placed over the portion of the user's body to receive treatment such that each opening 35 in device 10 receives and accommodates the one or more corresponding external portions of external fixation device 5. At step 106, one or more straps 18, 26 may be used to couple together opposing portions of device 10 to secure the device around the portion of the user's body. At step 108, one or more fluid-impermeable bladders 40 are at least partially filled with an appropriate fluid, such as air, water, or any other fluid. At step 110, device 10 is used to apply intermittent compression to the portion of the user's body in response to corresponding intermittent increases and decreases in fluid pressure in bladder 40. Such compression may be periodically, cyclically, or otherwise intermittently applied.

Although an example method is illustrated, the present invention contemplates two or more steps taking place substantially simultaneously or in a different order. In addition, the present invention contemplates using methods with additional steps, fewer steps, or different steps, so long as the methods remain appropriate for using a fluidic compression device 10 adapted to accommodate an external fixation device 5 to prevent or treat a user's circulation problems.

Furthermore, although the present invention has been described with several embodiments, a multitude of changes, substitutions, variations, alterations, and modifications may be suggested to one skilled in the art, and it is intended that the invention encompass all such changes, substitutions, variations, alterations, and modifications as fall within the spirit and scope of the appended claims.

What is claimed is:

1. A fluidic compression device adapted to accommodate an external fixation device, comprising:
at least one fluid-impermeable bladder adapted to contain a fluid; and
at least one region adapted to receive and accommodate one or more external portions of an external fixation device;
the device adapted to be positioned about a portion of a user's body such that an opening formed within the region receives and accommodates the one or more external portions of the external fixation device, the device further adapted to apply intermittent compression to the portion of the user's body according to intermittent increases and decreases in fluid pressure in the bladder while the device is positioned about the portion of the user's body and is accommodating the external fixation device.

2. The device of claim 1, further comprising a seal defining the region in which the opening is formed, the seal separating the opening from the bladder and protecting the bladder from being perforated when the opening is formed.

3. The device of claim 2, wherein the seal comprises a double seal.

4. The device of claim 1, wherein the opening may be cut to customize the device to accommodate the external fixation device.

5. The device of claim 4, further comprising one or more pre-formed holes adapted to receive a cutting device for cutting the opening.

6. The device of claim 4, further comprising stitching within the region in which the opening is formed, the stitching helping to ensure that the opening is cut without compromising the integrity of the bladder.

7. The device of claim 4, further comprising a visible cut line imprinted on the device within the region in which the opening is formed to provide a guide for cutting the opening to help prevent the bladder from being perforated during cutting.

8. The device of claim 1, further comprising a connector coupled to the bladder and adapted to receive the fluid from a fluid source.

9. The device of claim 8, wherein the connector comprises an integral valve operable to maintain a fluid pressure in the bladder.

10. The device of claim 1, further comprising one or more straps adapted to couple together opposing portions of the device to secure the device about the portion of the user's body.

11. The device of claim 10, further comprising an opening formed in a strap adapted to allow one or more external portions of the external fixation device to pass through the strap.

12. The device of claim 10, wherein the one or more straps comprise hook and loop material.

13. The device of claim 1, further comprising:
a soft, foldable, and flexible internal foam material substantially encompassing the bladder; and
a soft, foldable, and flexible external woven material substantially covering the internal foam material.

14. The device of claim 13, further comprising a rounded edge portion to improve comfort and prevent or reduce dermal ulcerations.

15. A method for manufacturing a fluidic compression device adapted to accommodate an external fixation device, comprising:
providing at least one fluid-impermeable bladder adapted to contain a fluid; and
providing at least one region adapted to receive and accommodate one or more external portions of an external fixation device;
such that the device is adapted to be positioned about a portion of a user's body such that an opening formed within the region receives and accommodates the one or more external portions of the external fixation device, and such that the device is further adapted to apply intermittent compression to the portion of the user's body according to intermittent increases and decreases in fluid pressure in the bladder while the device is positioned about the portion of the user's body and is accommodating the external fixation device.

16. The method of claim 15, further comprising providing a seal defining the region in which the opening is formed, the seal separating the opening from the bladder and protecting the bladder from being perforated when the opening is formed.

17. The method of claim 16, wherein the seal comprises a double seal.

18. The method of claim 15, wherein the opening may be cut to customize the device to accommodate the external fixation device.

19. The method of claim 18, further comprising providing one or more pre-formed holes adapted to receive a cutting device for cutting the opening.

20. The method of claim 18, further comprising providing stitching within the region in which the opening is formed, the stitching helping to ensure that the opening is cut without compromising the integrity of the bladder.

21. The method of claim 18, further comprising providing a visible cut line imprinted on the device within the region in which the opening is formed to provide a guide for cutting the opening to help prevent the bladder from being perforated during cutting.

22. The method of claim 15, further comprising providing a connector coupled to the bladder and adapted to receive the fluid from a fluid source.

23. The method of claim 22, wherein the connector comprises an integral valve operable to maintain a fluid pressure in the bladder.

24. The method of claim 15, further comprising providing one or more straps adapted to couple together opposing portions of the device to secure the device about the portion of the user's body.

25. The method of claim 24, further comprising providing an opening formed in a strap adapted to allow one or more external portions of the external fixation device to pass through the strap.

26. The method of claim 24, wherein the one or more straps comprise hook and loop material.

27. The method of claim 15, further comprising:
providing a soft, foldable, and flexible internal foam material substantially encompassing the bladder; and
providing a soft, foldable, and flexible external woven material substantially covering the internal foam material.

28. The method of claim 27, further comprising providing a rounded edge portion to improve comfort and prevent or reduce dermal ulcerations.

29. A method for preventing or treating circulation problems, comprising:
positioning a fluidic compression device about a portion of a user's body such that at least one opening formed in a region of the device receives and accommodates one or more external portions of an external fixation device;
at least partially filling at least one fluid-impermeable bladder of the device with fluid; and
intermittently increasing and decreasing fluid pressure within the bladder to apply intermittent compression to the portion of the user's body while the device is positioned about the portion of the user's body and is accommodating the external fixation device, the intermittent compression facilitating prevention or treatment of circulation problems associated with the portion of the user's body.

30. The method of claim 29, further comprising forming the opening within a sealed region of the device.

31. The method of claim 29, wherein the device further comprises a seal defining a region in which the opening is formed, the seal separating the opening from the bladder and protecting the bladder from being perforated when the opening is formed.

32. The method of claim 31, wherein the seal comprises a double seal.

33. The method of claim 29, wherein the opening may be cut to customize the device to accommodate the external fixation device.

34. The method of claim 33, wherein the device further comprises one or more pre-formed holes adapted to receive a cutting device for cutting the opening.

35. The method of claim 33, wherein the device further comprises stitching within a region in which the opening is formed, the stitching helping to ensure that the opening is cut without compromising the integrity of the bladder.

36. The method of claim 33, wherein the device further comprises a visible cut line imprinted on the device within a region in which the opening is formed to provide a guide for cutting the opening to help prevent the bladder from being perforated during cutting.

37. The method of claim 29, wherein the device further comprises a connector coupled to the bladder and adapted to receive the fluid from a fluid source.

38. The method of claim 37, wherein the connector comprises an integral valve operable to maintain a fluid pressure in the bladder.

39. The method of claim 29, further comprising using one or more straps to couple together opposing portions of the device to secure the device about the portion of the user's body.

40. The method of claim 39, wherein the device further comprises an opening formed in a strap adapted to allow one or more external portions of the external fixation device to pass through the strap.

41. The method of claim 39, wherein the one or more straps comprise hook and loop material.

42. The method of claim 29, wherein the device further comprises:
a soft, foldable, and flexible internal foam material substantially encompassing the bladder; and
a soft, foldable, and flexible external woven material substantially covering the internal foam material.

43. The method of claim 42, wherein the device further comprises a rounded edge portion to improve comfort and prevent or reduce dermal ulcerations.

44. A fluidic compression device adapted to accommodate an external fixation device, comprising:
first means for containing a fluid; and
second means for defining a region for receiving and accommodating one or more external portions of an external fixation device;
the device adapted to be positioned about a portion of a user's body such that an opening formed in the region receives and accommodates the one or more external portions of the external fixation device, the device further adapted to apply intermittent compression to the portion of the user's body according to intermittent increases and decreases in fluid pressure in the first means while the device is positioned about the portion of the user's body and is accommodating the external fixation device.

45. A fluidic compression device adapted to accommodate an external fixation device, comprising:
at least one fluid-impermeable bladder adapted to contain a fluid;
at least one sealed region comprising:
an opening cut to customize the device to receive and accommodate one or more external portions of an external fixation device, the seal separating the opening from the bladder and protecting the bladder from being perforated when the opening is cut;
one or more pre-formed holes adapted to receive a cutting device for cutting the opening; and
stitching helping to ensure that the opening is cut without compromising the integrity of the bladder;
the device adapted to be positioned about a portion of a user's body such that the opening cut within the sealed region receives and accommodates the one or more external portions of the external fixation device, the device further adapted to apply intermittent compression to the portion of the user's body according to intermittent increases and decreases in fluid pressure in the bladder while the device is positioned about the portion of the user's body and is accommodating the external fixation device.
